(19) United States
(12) Patent Application Publication
(10) Pub. No.: US 2009/0131749 A1
(43) Pub. Date: May 21, 2009

(54) TISSUE REMOVAL SYSTEM AND METHOD USING REINFORCED LOOP

(75) Inventors: Ijaz AHMED, Galveston, TX (US); Gottumukkala S. RAJU, League City, TX (US)

Correspondence Address: LOCKE LORD BISSELL & LIDDELL LLP;ATTN: IP DOCKETING, 600 TRAVIS, SUITE 3400, HOUSTON, TX, 77002-3095, US

(73) Assignee: BOARD OF REGENTS, THE UNIVERSITY OF TEXAS SYSTEM, Austin, TX (US)

(21) Appl. No.: 12/274,545

(22) Filed: Nov. 20, 2008

Related U.S. Application Data
(60) Provisional application No. 60/989,573, filed on Nov. 21, 2007.

(51) Int. Cl. A61B 1/018; A61B 17/22

(52) U.S. Cl. 600/106; 606/113

(57) ABSTRACT

The present disclosure provides a system and method for removing a tissue that extends from a tissue layer, such as a polyp, lesion, or organ. The disclosure provides a medical loop that can be tightened around the tissue extending from the tissue layer by using a one-way anchor and a flexible member to form a loop, the flexible member having ridges to interface with the anchor and secure the flexible member in a tightened position around the tissue. The ridges interface with the tissue to secure the loop in position on the extending tissue and reduce slippage of the loop off of the tissue after tightening the loop. A snare or other cutting instrument may be used to remove the tissue extending outward from the loop. An endoscopic system may be used to guide the medical loop and the cutting instrument to an appropriate location inside a body passage.

### CROSS REFERENCE TO RELATED APPLICATIONS

This application claims the benefit of U.S. Provisional Appl. No. 60/989,573, filed on Nov. 21, 2007, and is incorporated herein by reference.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO APPENDIX

Not applicable.

### BACKGROUND

1. Field of the Invention

The present disclosure relates to medical devices and procedures. More particularly, the disclosure relates to tissue removal medical devices and procedures.

2. Description of Related Art

The removal of polyps that extend above a tissue layer using an endoscope has far reaching implications for the medical field. Such procedures are faster and less expensive, and therefore more available to a greater number of patients and less cumbersome for surgeons. Much effort has been made in this field to develop procedures and instruments to effectively removal such polyps.

A recent publication, P. Katsinelos, et al., “Endoloop-assisted polypectomy for large pedunculated colorectal polyps,” Surg. Endosc (2006) 20: 1257-1261, illustrates a procedure. The publication describes using a colonoscope inserted into a patient's colon with a detachable endoloop system composed of an operating part to remove a polyp having an enlarged head and a smaller diameter stalk supporting the head to the underlying tissue layer. The operating part consists of a Teflon sheath 2.5 mm in diameter by 195 cm long, a stainless steel coil sheath 1.9 mm in diameter, a hook wire, and a handle. A silicon rubber stopper is described as to maintaining a tightness of the loop. The loop is retracted inside the Teflon sheath for insertion through an accessory channel of the colonoscope and maneuvered using the video screen of the scope to the polyp. The loop is extended and tightened around a base of the stalk by sliding the stopper along the loop, and then the loop is detached from the operating part. A diathermic snare is then used to sever the stalk of the polyp above the tightened loop using electrosurgical coagulation current. The polyp is captured using a basket catheter and gently extracted through the colon and out of the body.

However, the article admits that some patients have some complications including hematochezia, requiring the patients to be hospitalized and the hematochezia ceased and blood tests became stable. The polypectomy-induced bleeding at times required further procedures to control the bleeding including hemoclip placement or heater probe treatment. Importantly, the article also admits that in some patients the endoloop slipped off after the polypectomy and the affected area showed delayed bleeding after several days, requiring the patients to be treated with additional hemoclipping procedures. Finally, the article clearly states that postpolypectomy hemorrhage caused by current available instruments frequently is an unpredictable event that creates great anxiety and, like all complications, is best avoided.

Thus, there remains a need for an improved medical loop system and procedure.

### BRIEF SUMMARY

The present disclosure provides a system and method for removing a tissue that extends from a tissue layer, such as a polyp, lesion, or organ. The disclosure provides a medical loop that can be tightened around the tissue extending from the tissue layer by using a one-way anchor and a flexible member to form a loop, the flexible member having ridges to interface with the anchor and secure the flexible member in a tightened position around the tissue. The ridges interface with the tissue to secure the loop in position on the extending tissue and reduce slippage of the loop off of the tissue after tightening the loop. A snare or other cutting instrument may be used to remove the tissue extending outward from the loop. An endoscopic system may be used to guide the medical loop and the cutting instrument to an appropriate location inside a body passage.

The disclosure provides a system having a medical loop suitable for surrounding and securing a tissue extending outwardly from a tissue layer, comprising: an anchor comprising a one-way engagement receiver; and a flexible member coupled to the anchor, the flexible member having a first end secured to the anchor, and a second end adapted to pass through the anchor and to slidably engage the anchor to form a loop, the flexible member having ridges formed on a peripheral inside surface of the flexible member and adapted to engage the one-way engagement receiver to secure the flexible member through the anchor in position around the tissue and adapted to engage the ridges with the tissue, the inside surface being oriented toward a middle of an area bounded by the loop.

The disclosure also provides a medical system, comprising: an endoscope having a longitudinal channel; a cutting instrument adapted to be inserted through the endoscope opening; and a detachable medical loop, comprising: an anchor comprising a one-way engagement receiver; and a flexible member coupled to the anchor, the flexible member having a first end secured to the anchor, and a second end adapted to pass through the anchor and to slidably engage the anchor to form a loop, the flexible member having ridges formed on a peripheral inside surface of the flexible member and adapted to engage the one-way engagement receiver to secure the flexible member through the anchor in position around the tissue and adapted to engage the ridges with the tissue, the inside surface being oriented toward a middle of an area bounded by the loop.

The disclosure further provides a method of removing an extending tissue from a tissue layer, comprising: inserting an endoscope having a channel formed therein through an opening in a body to a location proximal to the extending tissue; inserting a detachable medical loop comprising an anchor having a one-way engagement receiver and a flexible member coupled to the anchor, the flexible member having a first end secured to the anchor, and a second end adapted to pass through the anchor and form the loop, the flexible member having ridges formed on a peripheral inside surface of the flexible member, the inside surface being oriented toward a middle of an area bounded by the loop; at least partially surrounding the extending tissue with the medical loop; pulling the second end of the flexible member to form a tightened condition of the loop around the extending tissue; securing the loop is the tightened condition with the one-way engagement receiver of the anchor coupled with the ridges of the flexible member; and engaging the extending tissue with the ridges when the loop is in the tightened condition.

### BRIEF DESCRIPTION OF THE DRAWINGS

While the concepts provided herein are susceptible to various modifications and alternative forms, only a few specific embodiments have been shown by way of example in the drawings and are described in detail below. The figures and detailed descriptions of these specific embodiments are not intended to limit the breadth or scope of the concepts or the appended claims in any manner. Rather, the figures and detailed written descriptions are provided to illustrate the concepts to a person of ordinary skill in the art as required by 35 U.S.C. §112. In this field, special and sometimes simple devices from the viewpoint of hindsight can yield major improvements in costs, time, or the ability to even perform a desired medical procedure.

FIG. 1 is a schematic diagram of an exemplary system of a medical loop.

FIG. 11A is a schematic cross-sectional diagram of a detail of an anchor of the medical loop, and a flexible member passing through the anchor.

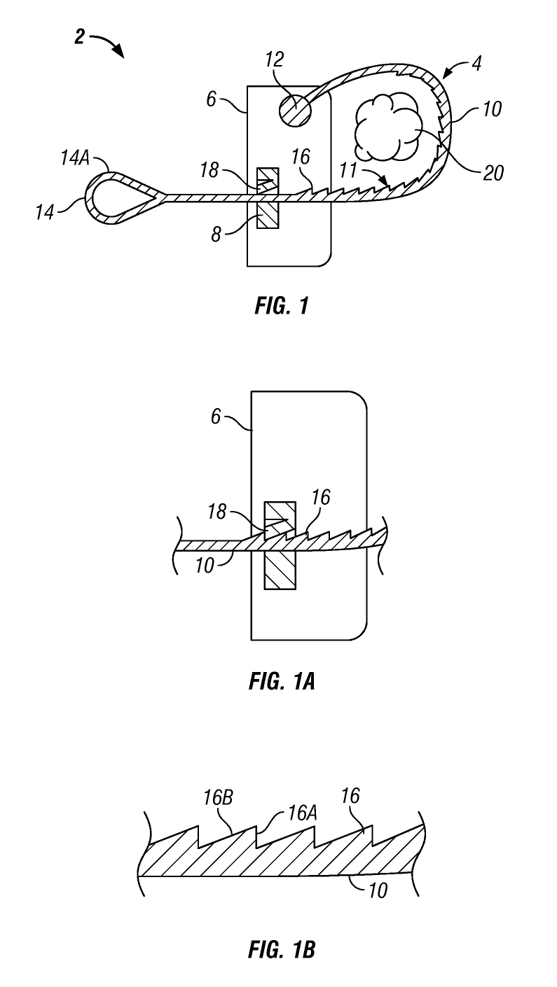

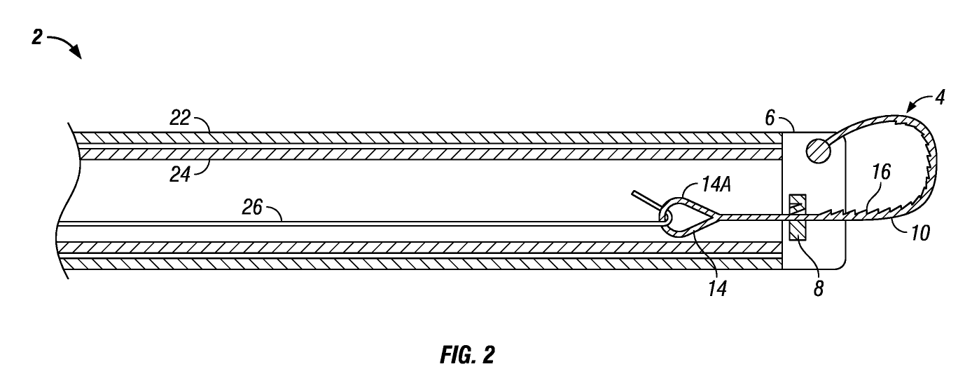

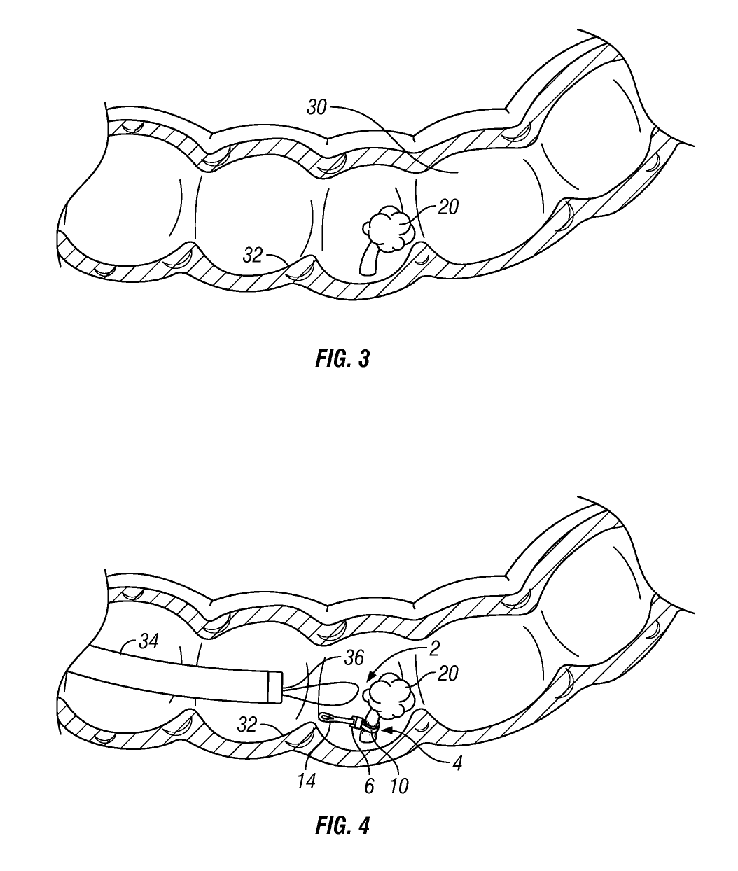

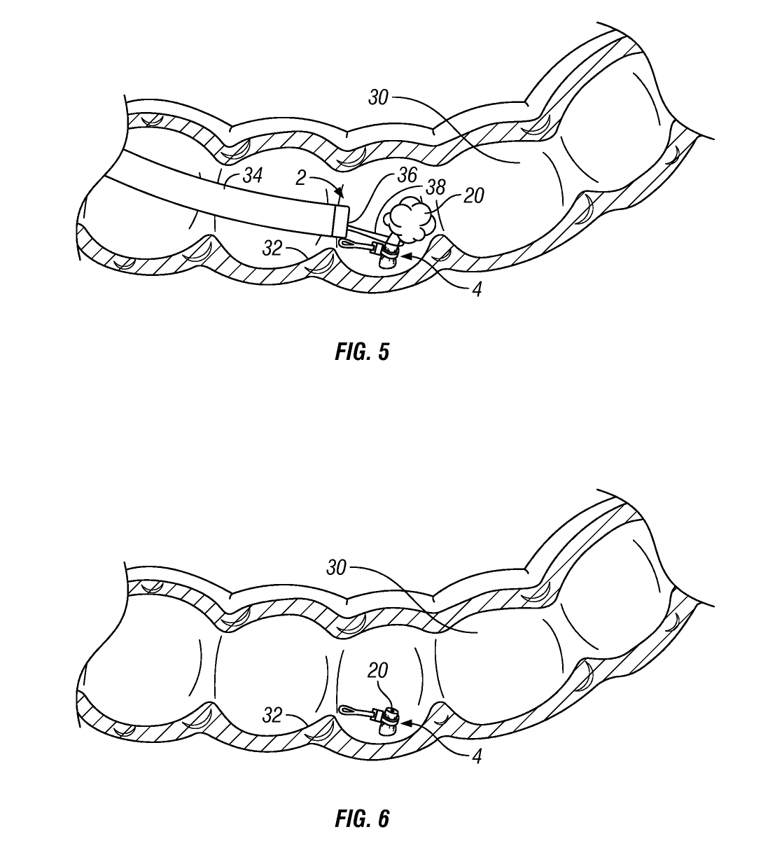

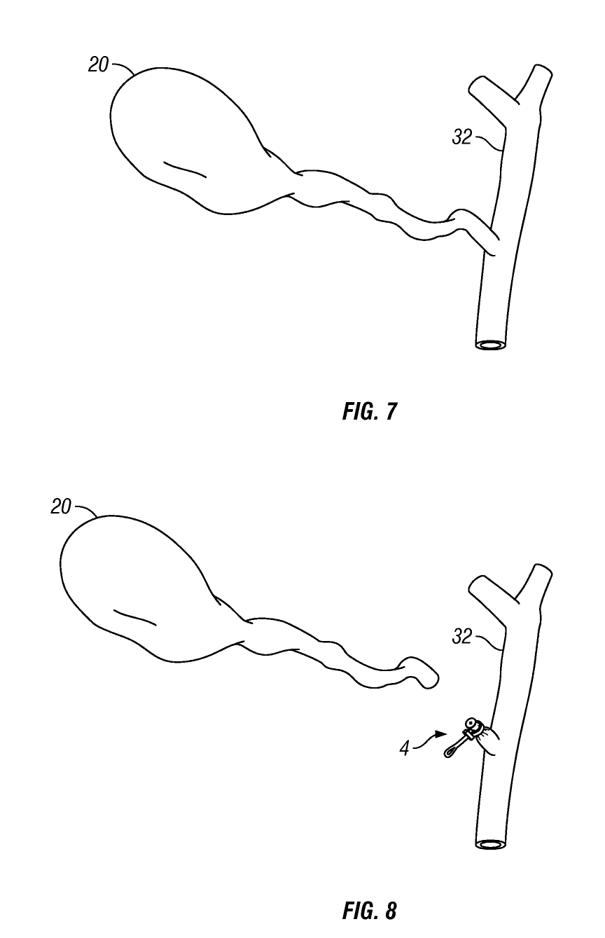

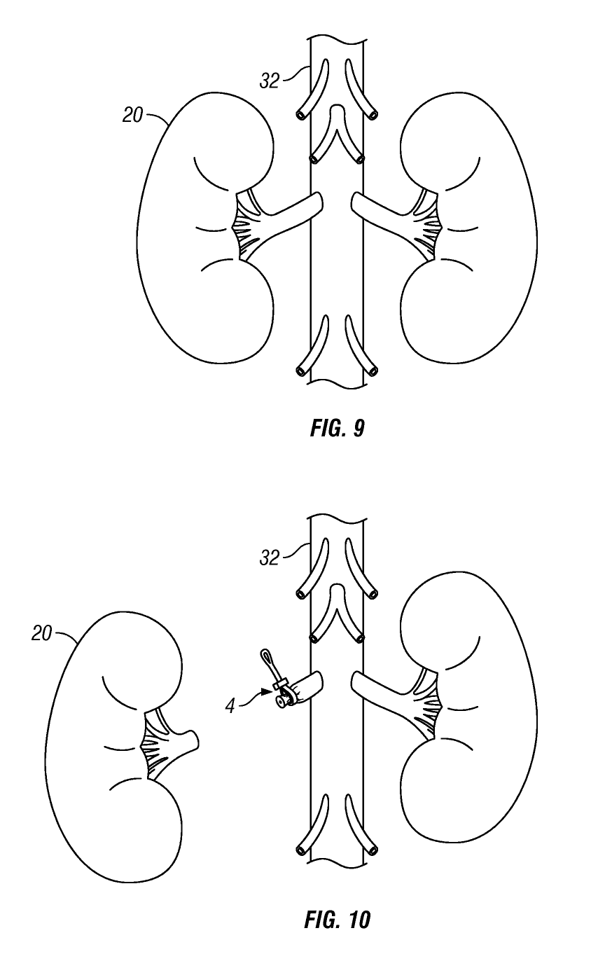

FIG. 1B is a schematic cross-sectional diagram of an exemplary detail of ridges on the flexible member.

FIG. 2 is a schematic cross-sectional diagram of an exemplary system having an outer and inner sheath coupled with the medical loop.

FIG. 3 is a schematic diagram of a body passage having a tissue extending outward from a tissue layer.

FIG. 4 is a schematic diagram of a body passage having an endoscope inserted therein with the medical loop tightened around the extending tissue.

FIG. 5 is a schematic diagram of a body passage having the endoscope inserted therein with the medical loop tightened around the tissue and a snare disposed around the tissue outwardly from the medical loop.

FIG. 6 is a schematic diagram of a body passage having at least a portion of the extending tissue excised from the tissue layer with the medical loop tightened around a remaining portion of the tissue.

FIG. 7 is another schematic diagram of a tissue in the form of an organ, extending from a tissue layer.

FIG. 8 is a schematic diagram of the tissue of FIG. 7 being removed from the tissue layer with the medical loop tightened around a remaining portion of the tissue.

FIG. 9 is another schematic diagram of a tissue in the form of an organ, extending from a tissue layer.

FIG. 10 is a schematic diagram of the tissue of FIG. 9 being removed from the tissue layer with the medical loop tightened around a remaining portion of the tissue.

### DETAILED DESCRIPTION

One or more illustrative embodiments of the concepts disclosed herein are presented below. Not all features of an actual implementation are described or shown in this application for the sake of clarity. It is understood that in the development of an actual embodiment, numerous implementation-specific decisions must be made to achieve the developer's goals, such as compliance with system-related, business-related and other constraints, which vary by implementation and from time to time. While a developer's efforts might be complex and time-consuming, such efforts would be, nevertheless, a routine undertaking for those of ordinary skill in the art having benefit of this disclosure.

FIG. 1 is a schematic diagram of an exemplary system of a medical loop. FIG. 1A is a schematic cross-sectional diagram of a detail of an anchor of the medical loop, and a flexible member passing through the anchor. FIG. 1B is an exemplary schematic cross-sectional diagram of a detail of ridges on the flexible member. The drawings will be described in conjunction with each other. A system 2 includes a medical loop 4. The medical loop 4 includes an anchor 6 having a receiver 8 coupled thereto. The medical loop 4 further includes a flexible member 10 having a first end 12 coupled to the anchor in a fixed location, and a second end 14 adapted to be slidably disposed within the receiver 8. The second end 14 can have a connection member 14A for attachment to various devices described herein. The flexible member 10 further comprises one or more ridges 16 formed on an inside surface 11 of the flexible member adapted to engage the receiver 8 as the flexible member 10 is inserted therethrough. The inside surface of the flexible member is oriented toward a middle of an area bounded by the loop when both ends of the flexible member are coupled to the anchor to form the loop. When the loop surrounds a tissue 20 disposed therein, the inside surface is also oriented toward the tissue.

The ridges 16 assist in one or more ways. First, the ridges 16 are adapted to be engaged by the receiver 8. The ridges 16 of the flexible member 10 can be inserted through the receiver 8, so that the ridges engage an engagement member 18 of the receiver 8. The engagement member 18 can be configured, so that the engagement member allows the flexible member 10 to more easily pass in one direction than in a reverse direction. Such embodiment is called herein a “one-way” embodiment. For example and without limitation, the engagement member 18 can be bendably coupled to a portion of the receiver 8. As the flexible member is inserted through the receiver, the engagement member 18 bends to allow the flexible member 10 to pass therethrough in a first direction, but resists bending if the flexible member tries to be extracted in the reverse direction. An example of such a configuration is shown in FIG. 1A. Still further, the ridges 16 can be formed from various shapes. In at least one exemplary embodiment and without limitation, the ridges 16 can include a first surface 16A and a second surface 1 6B, where the first surface 16A is more perpendicular to the length of the flexible member 10 than the second surface 16B. Other shapes, sizes, and configurations of the ridges 16 can be formed, and the described embodiment is only exemplary.

A further function of the ridges 16 on the inside surface of the flexible member 10 is to engage the tissue 20 extending from the tissue layer, described herein. In contrast to prior efforts that caused slippage off the tissue, the present disclosure provides that the ridges 16 affirmatively engage the tissue 20 at least when the medical loop 4 is tightened around the tissue. Such engagement assists in maintaining the medical loop in position around the tissue before and after excision of the tissue extending above the loop. For purposes herein, the “tissue” extending above the tissue surface includes various growths such as polyps, lesions, and other tissues that extend above a normal tissue layer that can be encircled by the medical loop. The tissue extending above the tissue surface further includes tissues of organs, vessels, and other tissues that need at least partial removal or constricting of fluid flow through the tissue. In most cases, such growth can be excised by a snare or other cutting instrument after the encircling and tightening of the medical loop around the tissue.

FIG. 2 is a schematic cross-sectional diagram of an exemplary system having an outer and inner sheath coupled with the medical loop. The system 2 having the medical loop 4 can further include one or more sheaths coupled with the medical loop for positioning of the medical loop around the tissue location. For example and without limitation, an outer sheath 22 can be formed around an inner sheath 24. Further, a loop control wire 26 can be inserted through the inner sheath 24 and coupled to the connection member 14A of the second end 14 of the flexible member 10. In at least one embodiment, the anchor 6 can be coupled to the outer sheath 22. The assembly can be disposed in a proximal location to the tissue and the loop control wire pulled so that the second end 14 of the flexible member 10 is pulled through the receiver 8 of the anchor 6, and tightened around the tissue as shown. When sufficiently tightened, the inner sheath 24 can be manipulated to push off, or otherwise release, the medical loop 4 from the outer sheath 22 and left in position around the tissue. The loop control wire 26 can be disconnected from the connection member 14A of the second end 14. The inner sheath and outer sheath can be retracted from the location to allow for any further procedures. Alternatively, the medical loop 4 can be coupled to the inner sheath 24, the medical loop tightened as described above, and the outer sheath 22 can be manipulated to push off, or otherwise release, the medical loop 4 from the inner sheath 24.

FIG. 3 is a schematic diagram of a body passage having a tissue extending outward from a tissue layer. A body passage 30, such as a colon, esophagus, or an incision through a portion of the body can include a tissue 20 extending outwardly from a tissue layer 32. By “outwardly,” it is meant that the tissue extends in a direction away from the normal tissue layer 32. For example and without limitation, a polyp normally has an enlarged “head” and is coupled to the tissue layer with a narrower “stalk.” Other shapes are contemplated by the present invention. Such tissues extending from the tissue layer can be indicative of abnormal growth and may need at least encirclement of a medical loop and tightening thereon with generally excision from the tissue layer 32.

FIG. 4 is a schematic diagram of a body passage having an endoscope inserted therein with the medical loop tightened around the extending tissue. The terms “endoscope” or “scope” are used broadly in this application and include any tool insertable into a body having a channel through which tools and other devices can be placed and used, whether inserted through a natural body orifice or through an artificially created opening, such as through an incision or other procedure (generally termed “body passage” herein), and thus includes other medical scopes modified to be able to convey tools and related instruments or for viewing of internal body tissues. Thus, for present purposes, references to endoscopes, and more generally scopes also include entero scopes, esophagoscopes, colonoscopies, laparoscopes, pediatric scopes, choledochoscopes, pancreato scopes, esophagogastroduodeno (EGD) scope, and so forth. Such endoscopes can be used in artificial openings in the body (including for example “Single Port Access” known as “SPA”), or in natural openings in the body (including for example “Natural Orifice Translumenal Endoscopic Surgery” known as “NOTES”).

In at least one embodiment, an endoscope can be used to gain access to the tissue 20 by inserting the endoscope through a body opening either naturally or artificially created. The endoscope can be guided as is known to those with ordinary skill in the art, using video cameras and other portions of the endoscope not specifically shown but included herein to the proper location of the tissue 20. The endoscope generally has one or more channels 36 longitudinally formed therein. Various instruments, such as those described herein, can be inserted through the one or more channels 36. In this embodiment, the channel 36 can be used to insert the medical loop 4 described in FIGS. 1-2 above to locate and tighten the loop 4 around the tissue 20. Generally, it is advantageous to tighten the loop 4 at a location close to the tissue layer 32, so that a majority of the extending tissue 20 can be treated or excised. The loop 4 can be tightened as described above by pulling on the second end 14 of the flexible member 10 through the receiver 8 of the anchor 6, and then released, so that the loop can stay in position around the tissue. In many cases, it may be advantageous to tighten the loop 4 to such a degree that the tissue 20 changes color indicating that blood flow has stopped or been restricted into the tissue portion. The tissue is surrounded with the medical loop in a ligated condition.

FIG. 5 is a schematic diagram of a body passage having the endoscope inserted therein with the medical loop tightened around the tissue and a snare disposed around the tissue outwardly from the medical loop. If the tissue is to be excised, a snare or other instrument suited for removal of tissue can be inserted through the one or more channels 36 of the endoscope 34 to remove the tissue. In the exemplary embodiment, a snare 38 is indicated, although other cutting instruments may be used. The snare 38 can be positioned to encircle the tissue 20 and then tightened, such that the tissue 20 may be excised through a transection. In some operations, depending on the size of the tissue, the excision can occur at one time. In other situations, the excision may need to occur in a piece-meal fashion.

FIG. 6 is a schematic diagram of a body passage having at least a portion of the extending tissue excised from the tissue layer with the medical loop tightened around a remaining portion of the tissue. The present disclosure provides that the medical loop 4 can remain engaged with the remaining portion of the tissue 20, and can resist slipping off of the tissue 20 using the ridges 16, shown in FIGS. 1-2. The medical loop 4 can thus reduce the bleeding and other complications known to the prior art. Further, the medical loop can provide a secure closure in addition to or in lieu of sutures. In time, the growth will generally heal and eventually slough off the medical loop 4 and be naturally disposed through the body. In other embodiments, the medical loop can be formed of bio-compatible material that will eventually dissolve in the body.

FIG. 7 is a schematic diagram of a tissue in the form of an organ, extending from a tissue layer. FIG. 8 is a schematic diagram of the tissue of FIG. 7 being removed from the tissue layer with the medical loop tightened around a remaining portion of the tissue. The figures will be described in conjunction with each other. As defined above, the tissue 20 can include organs. For example, a gall bladder as a tissue portion 20 is connected to a common hepatic duct as a tissue layer 32 through a cystic duct. For purposes herein, the cystic duct can be considered as part of the tissue 20. A surgeon can recommend removal of the gall bladder in a procedure known as a “cholecystectomy.” The medical loop 4 can be applied to the tissue from which the gall bladder is removed, which in this example would generally be a remaining portion of the cystic duct adjacent the tissue layer 32. The medical loop can provide advantageous anchoring and closure of the opening.

FIG. 9 is another schematic diagram of a tissue in the form of an organ, extending from a tissue layer. FIG. 10 is a schematic diagram of the tissue of FIG. 9 being removed from the tissue layer with the medical loop tightened around a remaining portion of the tissue. The figures will be described in conjunction with each other. As an example, a kidney as a tissue portion 20 is connected to an abdominal aorta as a tissue layer 32 through a renal artery. For purposes herein, the renal artery (and corresponding renal vein) can be considered part of the tissue 20 as vascular tissues. If a kidney is removed, the medical loop 4 can be applied to the portion of the vascular tissues remaining after the removal. The medical loop can provide advantageous anchoring and closure of the opening. Such flexibility can be used in similar procedures for other tissues, including organs, in the abdomen (including for example an appendectomy), thorax, brain, uterus (including for example a hysterectomy), and other locations.

The invention has been described in the context of various embodiments and not every embodiment of the invention has been described. Apparent modifications and alterations to the described embodiments are available to those of ordinary skill in the art. For example, the cutting instrument can be used to remove the tissue prior to the medical loop being placed over the tissue and/or tightened around the tissue. The disclosed and undisclosed embodiments are not intended to limit or restrict the scope or applicability of the invention conceived of by the Applicant, but rather, in conformity with the patent laws, Applicant intends to protect all such modifications and improvements to the full extent that such falls within the scope or range of equivalents of the following claims.

The various methods and embodiments of the invention can be included in combination with each other to produce variations of the disclosed methods and embodiments, as would be understood by those with ordinary skill in the art, given the understanding provided herein. Also, various aspects of the embodiments could be used in conjunction with each other to accomplish the understood goals of the invention. Also, the directions such as “top,” “bottom,” “left,” “right,” “upper,” “lower,” and other directions and orientations are described herein for clarity in reference to the figures and are not to be limiting of the actual device or system or use of the device or system. The term “coupled,” “coupling,” “coupler,” and like terms are used broadly herein and can include any method or device for securing, binding, bonding, fastening, attaching, joining, inserting therein, forming thereon or therein, communicating, or otherwise associating, for example, mechanically, magnetically, electrically, chemically, directly or indirectly with intermediate elements, one or more pieces of members together and can further include without limitation integrally forming one functional member with another in a unity fashion. The coupling can occur in any direction, including rotationally. Unless the context requires otherwise, the word “comprise” or variations such as “comprises” or “comprising”, should be understood to imply the inclusion of at least the stated element or step, or group of elements or steps, or equivalents thereof, and not the exclusion of a greater numerical quantity or any other element or step, or group of elements or steps, or equivalents thereof The device or system may be used in a number of directions and orientations. Further, the order of steps can occur in a variety of sequences unless otherwise specifically limited. The various steps described herein can be combined with other steps, interlineated with the stated steps, and/or split into multiple steps. Additionally, the headings herein are for the convenience of the reader and are not intended to limit the scope of the invention.

Further, any references mentioned in the application for this patent as well as all references listed in the information disclosure originally filed with the application are hereby incorporated by reference in their entirety, to the extent such may be deemed essential to support the enabling of the invention. However, to the extent statements might be considered inconsistent with the patenting of the invention, such statements are expressly not meant to be considered as made by the Applicants.

1. A system having a medical loop suitable for securing a tissue extending outwardly from a tissue layer, comprising:
an anchor comprising a one-way engagement receiver; and
a flexible member coupled to the anchor, the flexible member having a first end secured to the anchor, and a second end adapted to pass through the anchor and to slidably engage the anchor to form a loop, the flexible member having ridges formed on a peripheral inside surface of the flexible member and adapted to engage the one-way engagement receiver to secure the flexible member through the anchor in position around the tissue and adapted to engage the ridges with the tissue, the inside surface being oriented toward a middle of an area bounded by the loop.

2. The system of claim 1, further comprising an outer sheath and an inner sheath slidably disposed in the outer sheath, and the anchor coupled to at least one of the sheaths.

3. The system of claim 2, wherein the anchor is releasably coupled to outer sheath and the inner sheath is adapted to push off the anchor from the outer sheath.

4. The system of claim 2, wherein the anchor is releasably coupled to inner sheath and the outer sheath is adapted to push off the anchor from the inner sheath.

5. The system of claim 1, further comprising a loop control wire slidably disposed in the inner sheath and coupled to the second end and adapted to pull a portion of the flexible member through the anchor.

6. The system of claim 1, further comprising an endoscope having at least one channel wherein the medical loop is adapted to be slidably disposed therethrough.

7. A system for securing and removing a tissue extending outward from a tissue layer, comprising:
an endoscope having a longitudinal channel;
a cutting instrument adapted to be inserted through the endoscope channel; and
a detachable medical loop adapted to be inserted through the endoscope channel, comprising:
an anchor comprising a one-way engagement receiver; and
a flexible member coupled to the anchor, the flexible member having a first end secured to the anchor, and a second end adapted to pass through the anchor and to slidably engage the anchor to form a loop, the flexible member having ridges formed on a peripheral inside surface of the flexible member and adapted to engage the one-way engagement receiver to secure the flexible member through the anchor in position around the tissue and adapted to engage the ridges with the tissue, the inside surface being oriented toward a middle of an area bounded by the loop.

8. The system of claim 7, further comprising an outer sheath and an inner sheath slidably disposed in the outer sheath, and the anchor coupled to at least one of the sheaths.

9. The system of claim 8, wherein the anchor is releasably coupled to outer sheath and the inner sheath is adapted to push off the anchor from the outer sheath.

10. The system of claim 8, wherein the anchor is releasably coupled to inner sheath and the outer sheath is adapted to push off the anchor from the inner sheath.

11. The system of claim 7, further comprising a loop control wire slidably disposed in the inner sheath and coupled to the second end and adapted to pull a portion of the flexible member through the anchor.

12. A method of removing an extending tissue from a tissue layer, comprising:
inserting an endoscope having a channel formed therein through an opening in a body to a location proximal to the extending tissue;
inserting a detachable medical loop comprising an anchor having a one-way engagement receiver and a flexible member coupled to the anchor, the flexible member having a first end secured to the anchor, and a second end adapted to pass through the anchor and form the loop, the flexible member having ridges formed on a peripheral inside surface of the flexible member, the inside surface being oriented toward a middle of an area bounded by the loop;
at least partially surrounding the extending tissue with the medical loop;
pulling the second end of the flexible member to form a tightened condition of the loop around the extending tissue;
securing the loop is the tightened condition with the one-way engagement receiver of the anchor coupled with the ridges of the flexible member; and
engaging the extending tissue with the ridges when the loop is in the tightened condition.

13. The method of claim 12, further comprising ligating the extending tissue with the medical loop.

14. The method of claim 12, further comprising inserting a cutting instrument through the endoscope to the extending tissue.

15. The method of claim 14, further comprising excising at least a portion of the tissue extending beyond the medical loop distally from the tissue layer while the medical loop is in the tightened position.

16. The method of claim 14, further comprising excising at least a portion of the tissue at least prior to securing the loop around the tissue.
